(19) United States
(12) Patent Application Publication
(10) Pub. No.: US 2026/0000622 A1
(43) Pub. Date: Jan. 1, 2026

(54) NOVEL COATING PROTECTING ACTIVE COMPOUNDS FOR PHARMACEUTICAL, ENZYME, NUTRACEUTICAL, FOOD OR FEED APPLICATIONS

(71) Applicant: KEMIN INDUSTRIES, INC., DES MOINES, IA (US)

(72) Inventors: Massimiliano Donato Verona, Guastalla (IT); Bindu Lakshmibai Vasanthakumari, Waukee, IA (US); Ye Lao, West Des Moines, IA (US)

(73) Assignee: KEMIN INDUSTRIES, INC., DES MOINES, IA (US)

(21) Appl. No.: 19/256,093

(22) Filed: Jun. 30, 2025

Related U.S. Application Data
(60) Provisional application No. 63/665,317, filed on Jun. 28, 2024.

(51) Int. Cl. A61K 9/50

(52) U.S. Cl. CPC A61K 9/5047; A61K 9/5015

(57) ABSTRACT

The present invention relates to a unique coating comprising a hydrophobic matrix of fatty acids and a water-soluble weak organic acid. During acidic conditions (e.g., pH≤3), the weak organic acid is protonated and is almost insoluble or slowly solubilized, while at neutral to alkaline pH (e.g., pH 6 or higher) it is deprotonated and more easily solubilized, allowing for the release of the active ingredient. All of the materials in the coating are soluble in organic solvents and, preferably, alcohols. In at least one embodiment, the coating is used as an enteric coating. In at least one embodiment, the coating is evenly dispersed without the inclusion of a plasticizer, emulsifier, or emulsifying agent.

### CROSS-REFERENCE TO RELATED APPLICATIONS

The present application claims the benefit of priority to U.S. Patent Application No. 63/665,317, filed on Jun. 28, 2024, entitled “NOVEL ENTERIC COATING PROTECTING ACTIVE COMPOUNDS FOR PHARMACEUTICAL, NUTRACEUTICAL, FOOD OR FEED APPLICATIONS,” the entire disclosure of which is incorporated by reference herein in its entirety.

### BACKGROUND OF THE INVENTION

Enteric coatings are used on various tablets or pellets utilized to orally deliver a wide range of compounds, including pharmaceuticals, nutraceuticals, enzymes, and food or feed products. Enteric coatings are typically applied to protect pharmaceutical or nutraceutical compounds from being released and potentially inactivated by the low pH in the stomach. Commonly used enteric coatings comprise one or more of hydroxypropylmethylcellulose phthalate, cellulose acetate phthalate, carboxymethylethylcellulose, methyl methacrylate-methacrylate copolymer, methacrylate-ethyl acrylate copolymer, methacrylate-methyl acrylate-methyl methacrylate copolymer, hydroxypropylmethylcellulose acetate succinate, polyvinyl acetate phthalate and shellac.

These polymers were developed to achieve the desired protective qualities under acidic pH conditions and at the same time to release the active ingredient at a desired rate under an increased pH condition. The search for efficient natural alternatives to these polymers is impeded by the high cost and regulatory hurdles in using these coating materials for feed and food applications. While some natural alternatives to conventional enteric coatings have been tested for enteric resistance, oftentimes these alternatives are less desirable due to compromised acid protection, release properties or both.

For these and other reasons, there is a need for the present invention.

### SUMMARY OF THE INVENTION

The present invention relates to a unique coating for delivering active ingredients comprising a hydrophobic matrix of fatty acids and a water-soluble weak organic acid. In acidic conditions (e.g., pH≤3), the weak organic acid is protonated and its solubility limited, while at neutral to alkaline pH (e.g., pH 6 or higher) it is deprotonated and more easily solubilized, allowing for the release of the active ingredient. All of the materials in the coating are soluble in organic solvents and, preferably, alcohols. In general, the coating mixtures protect the dosage form from acidic degradation for at least an hour. In at least one embodiment, the coating is used enterically, but other applications are included within the scope of the invention.

The unique formulation of the invention provides the advantages of providing target acid stability of >85% and a fast release at neutral pH (>80% within 3 hours). It was further determined that acid stability and release were improved and/or retained through the addition of an additional heat-resistant coating.

### BRIEF DESCRIPTION OF THE DRAWINGS

FIG. 1 shows a schematic representation of the mode of action of the enteric coating.

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a,” “an,” and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising,” “including,” or “having” should be interpreted as referring to elements, components, or steps in a non-exclusive manner, so the referenced elements, components, or steps may be combined with other elements, components, or steps that are not expressly referenced. Embodiments referenced as “comprising,” “including,” or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive of their endpoints. For example, a numeric range of between 1 and 10 includes the values 1 and 10. When a series of numeric ranges are disclosed for a given value, the present disclosure expressly contemplates ranges including all combinations of the upper and lower bounds of those ranges. For example, a numeric range of between 1 and 10 or between 2 and 9 is intended to include the numeric ranges of between 1 and 9 and between 2 and 10.

The present disclosure provides a coating composition which allows for the release of the active ingredient after the stomach in the small intestine or in a lower part of the digestive tract. Another application is to use it as a protective layer against the acidity of the stomach. Once the ingredient passes the pyloric valve, it reaches the small intestine (or duodenum) having a pH of around 5. Depending on the pH solubility of the enteric coating, the active can already be released in this region of the digestive tract and be absorbed into the blood stream.

There are several primary reasons for which an enteric coating on an ingredient may be used, including: 1) protecting active pharmaceutical, nutraceutical, or other ingredients from the acidic environment of the stomach (e.g. enzymes and certain antibiotics); 2) preventing gastric distress or nausea from a drug due to irritation (e.g. sodium salicylate); 3) delivery of drugs that are optimally absorbed in the small intestine to their primary absorption site in their most concentrated form; 4) providing a delayed-release component for repeat action; 5) minimizing first pass metabolism of drugs; 6) preventing degradation inside the stomach by endogenous enzymes (e.g. pepsin). The most common drugs which cause stomach ulcers like aspirin, diclofenac, and naproxen are frequently available with enteric coatings.

During the coating process, a uniform protective layer is applied to the surface of the active in order to obtain the desired acidic resistance. In one embodiment of the invention, the coating provides acidic resistance of at least one hour. In one embodiment of the invention, coating provides acidic resistance of at least two hours.

Different equipment can be used to apply the coating depending upon the formulation. Usually when tablets and capsules are coated, a pan coater may be used, while in the case of pellets or granules a fluid bed may be used. The amount of coating to be applied to the active to achieve an optimal acidic resistance is defined by the weight gain linked to the surface area of the substrate. Due to the higher surface area of granules and microgranules compared to pellets/tablets/capsules, higher weight gain is needed for these solid dosage forms. For instance, while pellets, tablets, and capsules are coated to around 3-10% weight gain, granules and microgranules are coated to a weight gain of at least 15-30%.

One aspect of the present invention relates to a hydrophobic matrix and a pore forming compound. The hydrophobic matrix comprises at least one fatty acid having a chain length of between C14 to C22 and the pore-forming compound is at least one weak organic acid. As used here, the term “weak acid” refers to an organic acid that does not completely dissociate into its constituent ions when dissolved in solution and its dissociation ratio is dependent from its pka. The solubility of the pore-forming compound for use in the invention is pH dependent, wherein it has reduced solubility and is in protonated form while at low pH (≤3), while at neutral pH (6-8) to alkaline pH (>8) the compound is mainly in deprotonated form, allowing for faster dissolution in water. According to certain embodiments, the organic acid is selected from the group consisting of sorbic acid, benzoic acid, gallic acid, citric acid, lactic acid, and mixtures thereof.

A weight increase of 1% to 70% after application of the coating provides a coated dosage form having at least one of the properties described herein.

The hydrophobic matrix and the pore-forming compound can be present in a weight ratio of at least 1:2 and at most 20:1, including but not limited to, a weight ratio of at least 10:9, at least 9:8, at least 8:7, at least 7:6, at least 6:5, at least 5:4, at least 4:3, at least 3:2, at least 2:1, at least 3:1, or at least 4:1, or a weight ratio of at most 4:1, at most 35:9, at most 31:8, at most 27:7, at most 23:6, at most 19:5, at most 15:4, at most 11:3, at most 7:2, at most 3:1, or at most 2:1.

The hydrophobic matrix can be present in the dosage form coating composition in an amount by weight of at least 30.0% and at most 99.0%, including but not limited to, an amount by weight of at least 32.5%, at least 35%, at least 37.5.5%, at least 40%, at least 42.5%, at least 45.0%, at least 47.5%, at least 50.0%, at least 52.5%, at least 55.0%, at least 57.5%, at least 60.0%, at least 62.5%, at least 65.0%, at least 70.0%, at least 75.0%, at least 77.5%, at least 80.0%, at least 82.5%, at least 85.5%, at least 87.5%, at least 90.0%, at least 92.5%, at least 95.0%, at least 97.5%, or an amount by weight of at most 97.5%, at most 95.0%, at most 92.5%, at most 90.0%, at most 87.5%, at most 85.0%, at most 82.5%, at most 80.0%, at most 77.5%, at most 75.0%, at most 72.5%, at most 70.0%, at most 67.5%, at most 65.0%, at most 62.5%, at most 60.0%, at most 57.5%, at most 55.0%, at most 52.5%, at most 50.0%, at most 45.0%, or at most 42.5%.

The hydrophobic matrix can be one or more pH solubility independent and non-toxic fatty acids having a chain length between C10 to C25 that is also highly soluble in organic solvents, and preferably ethanol. In certain embodiments the hydrophobic matrix has a chain length of between C14 to C22. The fatty acids may be saturated, unsaturated, or polyunsaturated fatty acids or mixtures thereof. In the event of double bonds, both cis and trans configurations are suitable. Such compounds include, but are not limited to, lauric acid (water solubility 55 mg/L at 20° C.), myristic acid (water solubility 20 mg/L at 20° C.), palmitic acid (water solubility 7.2 mg/L at 20° C.), stearic acid (water solubility 2.9 mg/L at 20° C.), arachidic acid (totally insoluble), linoleic acid (water solubility 0.1 mg/L at 20° C.), erucic acid (totally insoluble). In general, a water solubility of below about 1 g/L (0.1%) is preferred for use in the invention. Stearic acid is a preferred hydrophobic matrix for use in the invention for purposes of cost.

The pore forming compound(s) can be present in the dosage form coating composition in an amount by weight of at least 1.0% and at most 60.0%, including but not limited to, an amount by weight of at least 2.0%, at least 5.0%, at least 7.5%, at least 10.0%, at least 12.5%, at least 15.0%, at least 17.5%, at least 20.0%, at least 22.5%, at least 25.0%, at least 27.5%, at least 30.0%, at least 32.5%, at least 35.0%, at least 37.5%, at least 40.0%, at least 42.5%, at least 45.0%, at least 47.5%, at least 50.0%, at least 52.5%, at least 55.0%, at least 57.5%, or an amount by weight of at most 57.5%, at most 55.0%, at most 52.5%, at most 50.0%, at most 47.5%, at most 45.0%, at most 42.5%, at most 40.0%, at most 37.5%, at most 35.0%, at most 32.5%, at most 30.0%, at most 29.0%, at most 28.0%, at most 27.0%, at most 26.0%, at most 25.0%, at most 22.5%, at most 20.0%, at most 17.5%, at most 15.0%, at most 12.5%, at most 10.0%, at most 7.5%, at most 5.0%, or at most 2.5%.

The pore forming compound(s) of the invention are weak organic acids that may be alkyl, alkenyl, and/or aryl carboxylic acids that are soluble in organic solvents. In one embodiment, the organic acids are soluble in alcohols. In one embodiment of the invention, the pore forming compound(s) are monocarboxylic compounds. In another embodiment of the invention, the pore forming compound(s) is a dicarboxylic, tricarboxylic, and/or polycarboxylic acid. In one embodiment of the invention, the pore forming compound(s) are one or more of sorbic acid, benzoic acid, citric acid, lactic acid, and/or gallic acid.

In some cases, the amount of hydrophobic matrix and pore forming compound(s) meet either the ratio limitations disclosed herein or the percent by weight values disclosed herein, and in some cases, they meet both.

In one embodiment of the invention, the hydrophobic matrix/pore forming layers of the dosage form coating composition are stearic acid/sorbic acid or stearic acid/benzoic acid. In one embodiment of the invention, the dosage form coating composition includes an additional heat-resistant layer comprising hydroxypropyl methylcellulose (HPMC) that is applied to the active ingredient prior to the application of the coating of the invention. In one embodiment of the invention, the heat-resistant layer may include one or more compounds that include, but are not limited to, ethyl cellulose, carboxymethyl cellulose, beeswax, microcrystalline wax, gelatin, modified starches, or similar. In an embodiment, the heat-resistant layer is placed outside the coating. The heat-resistant layer should generally withstand heat of between about 85-95° C.

The dosage form coating composition may optionally include a plasticizer. The plasticizer can be present in an amount by weight of at least 0.5% and at most 30.0%, including but not limited to, an amount by weight of at least 2.5%, at least 5.0%, at least 7.5%, at least 10.0%, at least 12.5%, at least 15.0%, at least 20.0%, or at least 25.0%, or an amount by weight of at most 25.0%, at most 20.0%, at most 17.5%, at most 15.0%, or at most 10.0%. The plasticizer can be any plasticizer that does not significantly alter the coating properties of the compositions, including for instance the enteric coating properties. The type of plasticizer and amount can be selected based on the desired viscosity of a resulting coating mixture. In some cases, the plasticizer can be glycerine (also called glycerol or glycerin). According to at least one embodiment, the coating composition does not include a plasticizer.

The dosage form coating composition may optionally include an emulsifier. The emulsifier can optionally be present in an amount by weight of at least 0.1% and at most 10.0%, including but not limited to, an amount by weight of at least 0.5%, at least 1.0%, at least 1.5%, at least 2.0%, at least 2.5%, at least 3.0%, at least 4.0%, at least 5.0%, or at least 7.5%, or an amount by weight of at most 9.0%, at most 8.0%, at most 7.0%, at most 6.0%, at most 5.0%, at most 4.0%, or at most 2.5%. The emulsifying agent can be any emulsifying agent that does not significantly alter the coating properties of the compositions. According to at least one embodiment, the coating composition does not include an emulsifier or emulsifying agent.

In one embodiment of the invention, the dosage form coating composition does not include a plasticizer nor an emulsifier. Plasticizers, such as triethyl citrate, dibutyl sebacate, and diethyl phthalate, are often included in enteric coatings in order to aid in adhesion of the coating to tablet's surface during the manufacturing process. A unique feature of the current coating composition is that it does not require the presence of a plasticizer and/or an emulsifier in order to ensure even distribution of the coating.

The dosage form coating composition can include an opacifying agent. The opacifying agent can be selected from the group consisting of titanium dioxide, calcium carbonate, Sensient® Avalanche™ (available commercially from Sensient Colors LLC, St. Louis, Mo.), other ingredients rendering opacification, and combinations thereof.

The dosage form coating composition can optionally include a sweetening agent. The sweetening agent can be selected from the group consisting of a sugar alcohol, an artificial sweetener, a natural sweetener, a sugar, and combinations thereof. The sugar alcohol can be selected from the group consisting of sorbitol, mannitol, xylitol, isomalt, hydrogenated starch hydrolysates, and combinations thereof. The artificial sweetener can be selected from the group consisting of sucralose, acesulfame, aspartame, and combinations thereof. The natural sweetener may include stevia. The sugar can be selected from the group consisting of sucrose, fructose, and combinations thereof.

The dosage form coating composition can include a flavorant. The flavorant can be a spray dried flavorant, a dried crystal flavorant, a granule flavorant, a liquid flavorant, or a combination thereof. The spray dried flavorant, the dried crystal flavorant, the granule flavorant, the liquid flavorant, or the combination thereof can comprise a synthetic flavoring agent, an artificial flavoring agent, a natural flavoring agent, or a combination thereof. The spray dried flavorant, the dried crystal flavorant, the granule flavorant, the liquid flavorant, or the combination thereof can provide a flavor selected from the group consisting of almond, amaretto, apple, green apple, apple-cherry-berry, apple-honey, apricot, bacon, banana, barbeque, beef, roast beef, beef steak, berry, berry blue, birch beer, spruce beer, blackberry, bloody mary, blueberry, boysenberry, brandy, bubble gum, butter, butter pecan, buttermilk, butterscotch, candy corn, cantaloupe, cantaloupe lime, caramel, carrot, cassia, caviar, celery, cereal, champagne, cherry, cherry cola, cherry maraschino, wild cherry, black cherry, red cherry, cherry-cola, chicken, chocolate, chocolate almond, cinnamon spice, citrus, citrus blend, citrus-strawberry, clam, cocoa, coconut, toasted coconut, coffee, coffee almond, cola, cola-vanilla, cookies & cream, cotton candy, cranberry, cranberry-raspberry, cream, cream soda, dairy type cream, creme de menthe, cucumber, black currant, dulce de leche, egg nog, pork fat, non-pork fat, anchovy fish, herring fish, sardine fish, frankfurter, fried garlic, sauteed garlic, gin, ginger ale, ginger beer, graham cracker type, grape, grape grapefruit, grapefruit-lemon, grapefruit-lime, grenadine, grill, guarana, guava, hazelnut, honey, roasted honey, ice cream cone, jalapeno, key lime, kiwi, kiwi-banana, kiwi-lemon-lime, kiwi-strawberry, kola champagne, lard type, lemon, lemon custard, lemonade, pink lemonade, lemon-lime, lime, malt, malted milk, mango, mango-pineapple, maple, margarita, marshmallow, meat type, condensed milk, cooked milk, mint, mirepoix, mocha, mochacinna, molasses, mushroom, sauteed mushroom, muskmelon, nectarine, neapolitan, green onion, sauteed onion, orange, orange cordial, orange creamsicle, orange creme, orange peach mango, orange strawberry banana, creamy orange, mandarin orange, orange-passion-guava, orange-pineapple, papaya, passion fruit, peach, peach-mango, peanut, roasted peanut, pear, pecan danish, pecan praline, pepper, peppermint, pimento, pina colada, pina colada/pineapple-coconut, pineapple, pineapple-orange, pistachio, pizza, pomegranate, baked potato, prune, punch, citrus punch, tropical punch, cherry fruit punch, grape punch, raspberry, black raspberry, blue raspberry, red raspberry, raspberry-blackberry, raspberry-ginger ale, raspberry-lime, root beer, rum, sangria, sarsaparilla, sassafras, sausage, sausage pizza, seafood, shrimp, hickory smoke, mesquite smoke, sour, sour cream, sour cream and onion, spearmint, strawberry, strawberry margarita, jam type strawberry, strawberry-kiwi, burnt sugar, tallow, tamarind, tangerine-lime, tangerine, tea, tequila, toffee, triple sec, tropical fruit mix, turkey, tutti frutti, vanilla, vanilla cream, vanilla custard, french vanilla, vegetable, vermouth, vinegar, balsamic vinegar, watermelon, whiskey, wildberry, wine, yogurt, and combinations thereof. The flavors described herein can be used alone or in combination with sensates described herein for experiential sensations of cooling, heating and tingling effects, such as use in combination with Sensient® Smoothenol® products.

The dosage form coating composition can include a sensate. The sensate can be a spray dried sensate, a dried crystal sensate, a granule sensate, a liquid sensate, or a combination thereof. The spray dried sensate, the dried crystal sensate, the granule sensate, the liquid sensate, or a combination thereof can provide a hot sensation, a cool sensation, a tingling sensation, or a combination thereof. In some cases, the sensate can be combined with a flavorant to provide a combination flavorant and sensate that combines the flavors and the sensations disclosed herein. In the event that a flavorant is combined with a sensate, the combination flavorant and sensate should be present in an amount that is equal to the amounts described herein with respect to flavorants and sensates.

The dosage form coating composition can include a flavor masking agent. The flavor masking agent can be selected from the group consisting of Smoothenol®, Smoothenol 2G® or numerical G Smoothenol®; such as 3G, 4G and in the forms of BitterFix™, AstringentFix™ FunctionalFix™, BurnFix™, SourFix™ (all available commercially from Sensient Flavors LLC, Hoffman Estates, Ill.), and combinations thereof. In some cases, the flavor masking agent can be combined with a flavorant, a sweetener, a sweetener enhancer, or the like. In some cases, the flavor masking agent can be contained in a combination product, such as Mafco's Magnasweet® line of products (available commercially from MAFCO Worldwide LLC, Camden, N.J.).

The dosage form coating composition can include a colorant. The colorant can be selected from the group consisting of a pigment, a dye, an exempt colorant (i.e., a colorant from a natural source), and combinations thereof.

The dosage form coating composition may also contain one or more of the following: binders such as gum tragacanth, acacia, corn starch or gelatin; excipients such as dicalcium phosphate; a disintegrating agent such as corn starch, potato starch, alginic acid and the like; or a lubricant such as magnesium stearate. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. Of course, any material used in preparing any unit dosage form should be pharmaceutically or food or feed acceptable, depending on the field of application, and substantially non-toxic in the amounts employed.

In one embodiment, the dosage form coating composition can be substantially free of various components that are commonly used in the dosage form enteric coating arts. The dosage form coating composition can be substantially free of methyl acrylate copolymers, acrylate copolymers, or a combination thereof, such as methyl acrylate-acrylate copolymer. The dosage form coating composition can be substantially free of methyl acrylate emulsion. The dosage form coating composition can be substantially free of polymer phthalates, such as cellulose acetate phthalate (CAP), hydroxypropyl methyl cellulose phthalate (HMPCP), polyvinyl acetate phthalate (PVAP), or combinations thereof. The dosage form coating composition can be substantially free of cellulose acetate phthalate (CAT). The dosage form coating composition can be substantially free of cellulosic film formers, such as hydroxypropyl methyl cellulose (HPMC), ethyl cellulose, carboxymethyl cellulose (CMC), sodium carboxymethyl cellulose (NaCMC), methyl cellulose, or combinations thereof. The dosage form coating composition can be substantially free of shellac. The dosage form coating composition can be substantially free of modified food starch or modified plant starch.

The present disclosure provides a dosage form coating mixture. The dosage form coating mixture can include the dosage form coating composition, as described elsewhere herein, and a solvent.

The dosage form coating and/or dosage form coating mixture can have a solids content of at least 5.0%, at least 10.0%, at least 20.0%, at least 30.0%, at least 40.0%, at least 50.0%, at least 60.0%, at least 70.0%, or at most 80.0%, at least 8.5%, at least 9.0%, at least 9.5%, at least 10.0%, at least 10.5%, or at least 11.0%.

The dosage form coating can have a viscosity of between 10 cP and 2000 cP, including but not limited to, between 500 cP and 1000 cP. In some cases, the viscosity can be at least 10 cP, at least 100 cP, at least 200 cP, at least 250 cP, at least 300 cP, at least 375 cP, at least 400 cP, at least 450 cP, or at least 500 cP. In some cases, the viscosity can be at most 2000 cP, at most 1750 cP, at most 1500 cP, at most 1350 cP, at most 1250 cP, at most 1200 cP, at most 1100 cP, at most 1000 cP, at most 900 cP, at most 850 cP, at most 750 cP, at most 600 cP, or at most 500 cP. In some cases, the viscosity can be represented as the viscosity measured at 10% solids (i.e., two coating mixtures formed from the same coating composition but with different solids contents would have the same viscosity). In other cases, the viscosity can be represented as the viscosity measured at the specific solids content of the coating mixture (i.e., two coating mixtures formed from the same coating composition but with different solids contents would have different viscosities).

The present disclosure provides a coating. The coating is the result of applying the dosage form coating mixture to an article in accordance with the methods described herein. The coating can include the same or substantially similar components as described elsewhere herein with respect to the dosage form coating composition, minus any volatile components that are removed in the coating process, as would be understood by a person having ordinary skill in the art.

The present disclosure provides a coated dosage form. The coated dosage form is the result of applying the dosage form coating mixture to a dosage form in accordance with the methods described herein. The coated dosage form includes the dosage form and the coating, as described elsewhere herein. The composition can be used to coat a wide variety of dosage forms, including but not limited to, tablets, granules, microgranules, pellets, mini-tablets, caplets, capsules, softgels, dissolvable strips, multiparticulates, and the like. In one embodiment, the dosage form is a granule having a diameter of less than 2.5 mm. In one embodiment, the dosage form is a microgranule having a diameter of less than 1.5 mm.

The dosage form coating may be used to coat any active ingredient that is compatible with the ingredients of the dosage form coating. In one embodiment of the invention, the dosage form coating is used to coat one or more acidic, alkaline, and/or neutral enzymes. In one embodiment, the enzymes are in granular form.

Persons of ordinary skill would appreciate that regulatory-friendly enteric coating development, in particular for food and feed applications, is challenging because it must satisfy multiple requirements, including: (i) use material approved for the target application, (ii) ensure a high acid protection (>85%), (iii) allow a fast active ingredient release at neutral pH (>80% within 3 h), (iv) withstand mechanical stress related to the coating application, and (v) cost effectiveness. In particular applications, and in particular for utilization in feed, the coating should also be robust for additional heat-resistant coating and survive pelleting conditions.

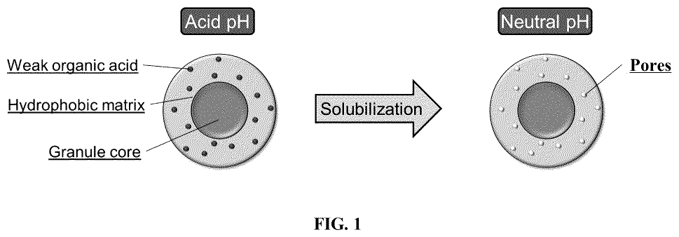

As noted, the coating compositions of the present invention comprise a hydrophobic matrix, with good filming properties and plasticity, and a novel pore former, based on weak organic acids. A schematic representation is depicted in FIG. 1.

The present disclosure provides a method of making a dosage form coating composition and/or mixture.

According to at least one embodiment, the coating composition is suitable for a solid dosage form, and comprises a first coating composition which comprises a hydrophobic matrix comprised of one or more fatty acids and a pore-forming compound comprising one or more weak organic acids, wherein the weak organic acid is protonated at a pH of about 3 or lower, and further wherein the weak organic acid is deprotonated at a pH of about 6 or higher, allowing for the release of the active ingredient that is coated.

The method of making the dosage form composition can include combining and/or mixing the various components of the dosage form coating composition.

The method of making the coating composition of the present invention comprises the following steps: 1) stirring a desired amount of solvent at a level sufficient to generate a vortex; 2) adding a desired amount of the dosage form coating composition; 3) mixing until a solution forms; and 4) optionally heating the solution until complete solubilization.

The present disclosure provides a method of using a dosage form coating composition for coating a solid dosage form. The solid dosage form may include any active ingredient or base component, including but not limited to a pharmaceutical, a nutraceutical, or a food or feed product, including but not limited to a food or feed additive.

In cases where the dosage form coating composition is the starting material, the method of using the dosage form coating composition can include preparing a dosage form coating mixture having a solids content as described elsewhere herein. The method can then continue with the method described below with respect to the dosage form coating mixture.

In cases where the dosage form coating composition is the starting material, the method of using the dosage form coating mixture can include applying the dosage form coating to a plurality of uncoated dosage forms. As used herein, “uncoated” refers to a dosage form that has not had this specific coating applied. It is possible that a preliminary or base coating is applied to the dosage form prior to applying the dosage form coating mixture such as a sealing coating like Opadry and Opadry II (available commercially from Colorcon). In one embodiment, the application can be by continuous drying technology or by fluid bed technology or pan-coating technology, or by any other application methods known in the art. Such methods are well understood by persons skilled in the art. The dosage forms may be any useful dosage form in the human, veterinary, and/or pet industries.

For purposes of tablets or pellets, the method can include applying the dosage form coating to a weight gain of at least 3%, at least 4%, or at least 5% and at most 10%, at most 9%, at most 8%, at most 7%, at most 6%, or at most 5%. For purposes of granules, the method can include applying the dosage form coating to a weight gain of at least 10%, at least 20%, at least 30%, at least 40%, at least 50%, at least 60%, at least 70%, or at most 80%. It should be appreciated that the methods of using the dosage form coating composition describe a broader range of weight gains than the weight gain range described with respect to the enteric properties achievable by the compositions and coatings described herein.

### EXAMPLES

The acid stability and the neutral release of the novel coating may be assessed according to multiple methods known by persons skilled in the art. For testing the products reported in the following examples a heated incubator shaker was used (WNB29, Memmert GmbH+Co. KG, Germany) set at 37° C. Acid resistance was carried out for 1 h at pH 2.5 (0.1M phosphate solution) at 80 spm. Neutral release was carried out by changing the pH to 7 and shaking at 160 spm. The concentration of the released active ingredient, at acid and neutral pH, was determined with a proper analytical method for each compound reported in the examples.

### Example 1

Control 1a: Uncoated enzymes granules, containing a protease, were produced from powder using a wet granulation technology. The average diameter was about 417 μm. Granules were tested for their acid stability without any coating. The enzyme activities were measured using Azocaein as substrate (S-AZCAS, Megazyme Itd). The granules were completely dissolved within 15 min. Acid resistance: 0%.
Sample 1a: A mixture of benzoic acid and fatty acids with a melting point (Mp) of 56-57° C. and a ratio of 1:9 was dissolved in hot ethanol. The solution was sprayed on 600 g of enzyme granules, as described in Control 1, until reaching a weight gain of 54%. The process was carried out using a fluid bed with an air temperature between 25 and 45° C. After spraying, the granules were further dried at 30-50° C. for about 5-10 min until obtaining a final moisture below 5%. Acid resistance: 100%. 2 h neutral release: 95%.
Sample 1b: A mixture of sorbic acid and fatty acids with a melting point (Mp) of 56-57 C° and a ratio of 1:9 was dissolved in hot ethanol. The solution was sprayed on 600 g of enzyme granules, as described in Control 1, until reaching a weight gain of 54%. The process was carried out using a fluid bed with an air temperature between 25 and 45° C. After spraying, the granules were further dried at 30-50° C. for about 5-10 min until obtaining a final moisture below 5%. Acid resistance: 100%. 2 h neutral release: 91%.
Sample 1c: 750 g of enzyme granules prepared according to Sample 1a and 1b were coated with a water dispersion of HPMC, talc and calcium stearate, with a ratio of 4.9:1:1, until reaching a weight gain of 2.8%. The coating was performed with a fluid bed with air between 35 and 50° C. The product was further dried for 10 min to reach a moisture below 5%. Acid resistance: 93%. 2 h neutral release: 82%.

### Example 2

Control 2: Uncoated methionine pellets were produced from powder using an extrusion technology. The average diameter was 2060 μm. Pellets were tested for their acid stability without any coating. The methionine concentration was measured with a HPLC method. Acid resistance: 13%.
Sample 2a: 1650 g of methionine pellets as described in Control 2 were coated with an ethanolic solution of sorbic acid and fatty matrix, with a ratio 1:9, until reaching a weight gain equal to 5%. The process was carried out using a fluid bed with an air temperature between 25 and 45° C. After spraying, the granules were further dried at 30-50° C. for about 5-10 min until obtaining a final moisture below 5%. Acid resistance: 99%. 3 h neutral release: 87%.
Sample 2b: 1000 g of methionine pellets as described in Control 2 were coated with an ethanolic solution of succinic acid and fatty matrix, with a ratio 1:1, until reaching a weight gain equal to 11%. The process was carried out using a fluid bed with an air temperature between 25 and 45° C. After spraying, the granules were further dried at 30-50° C. for about 5-10 min until obtaining a final moisture below 5%. Acid resistance: 93%. 2 h neutral release: 93%

### Example 3

Control 3: Uncoated lysine pellets were produced from powder using an extrusion technology. The average diameter was 2168 μm. Pellets were tested for their acid stability without any coating. Lysine concentration was determined with a HPLC method. Acid resistance: 4%.
Sample 3a: 1675 g of the lysine pellets described in Control 3 were coated with an ethanolic solution of gallic acid and fatty matrix, with a ratio 5:95, until reaching a weight gain equal to 8%. The process was carried out using a fluid bed with an air temperature between 25 and 45° C. After spraying, the granules were further dried at 30-50° C. for about 5-10 min until obtaining a final moisture below 5%. Acid resistance: 93%. 2 h neutral release: 94%.
Sample 3b: 600 g of the lysine pellets described in Control 3 were coated with an ethanolic solution of malic acid and fatty matrix, with a ratio 15:85, until reaching a weight gain equal to 11%. The process was carried out using a fluid bed with an air temperature between 25 and 45° C. After spraying, the granules were further dried at 30-50° C. for about 5-10 min until obtaining a final moisture below 5%. Acid resistance: 95%. 3 h neutral release: 89%.
Sample 3c: 600 g of the lysine pellets described in Control 3 were coated with an ethanolic solution of citric acid and fatty matrix, with a ratio 15:85, until reaching a weight gain equal to 12%. The process was carried out using a fluid bed with an air temperature between 25 and 45° C. After spraying, the granules were further dried at 30-50° C. for about 5-10 min until obtaining a final moisture below 5%. Acid resistance: 97%. 2 h neutral release: 90%.
Sample 3d: 600 g of the lysine pellets described in Control 3 were coated with an ethanolic solution of lactic acid and fatty matrix, with a ratio 15:85, until reaching a weight gain equal to 11%. The process was carried out using a fluid bed with an air temperature between 25 and 45° C. After spraying, the granules were further dried at 30-50° C. for about 5-10 min until obtaining a final moisture below 5%. Acid resistance: 99%. 2 h neutral release: 97%.
Sample 3e: 600 g of the lysine pellets described in Control 3 were coated with an ethanolic solution of sorbic acid and fatty matrix with a melting point (Mp) of 61-64 C.° with a ratio 15:85, until reaching a weight gain equal to 15%. Acid resistance: 96%. 3 h neutral release: 95%.
Sample 3f: 600 g of the lysine pellets described in Control 3 were coated with an ethanolic solution of sorbic acid and fatty matrix with a melting point (Mp) of 68-70 C° with a ratio 15:85, until reaching a weight gain equal to 14%. Acid resistance: 95%. 2 h neutral release: 83%.
Sample 3g: 600 g of lysine pellets of Control 3 were coated with an ethanolic solution of a fatty matrix with a melting point (Mp) of 56-57 C° without pore former, until reaching a weight gain equal to 10%. Acid resistance: 51%. 2 h neutral release: 100%
Sample 3h: 600 g of lysine pellets of Control 3 were coated with an ethanolic solution of a fatty matrix with a melting point (Mp) of 56-57 C° without pore former, until reaching a weight gain equal to 20%. Acid resistance: 97%. 2 h neutral release: 16% Example 4

A combination of ethyl cellulose and organic acid was used instead of the coating formula herein revealed. It should be appreciated that even by changing the matrix herein disclosed, it was not possible to meet both acid protection and neutral release.
Sample 4a: A mixture of sorbic acid and ethyl cellulose was dissolved in hot ethanol with a ratio 3:7. Oleic acid was used as plasticizer for ethyl cellulose with a ratio 1:8.2. The solution was sprayed on 300 g of enzyme granules, prepared according to Control 1, until reaching a weight gain of 25%. The process was carried out using a fluid bed with an air temperature between 25 and 45° C. After spraying, the granules were further dried at 30-50° C. for about 5-10 min until obtaining a final moisture below 5%. Acid resistance: 83%. 2 h neutral release: 57%.
Sample 4b: 300 g of enzyme granules of Control 1 were coated with an ethanolic solution of sorbic acid and ethyl cellulose, with a ratio 6:4, until reaching a weight gain equal to 25%. Oleic acid was used as plasticizer for ethyl cellulose with a ratio 1:8.2. The process was carried out using a fluid bed with an air temperature between 25 and 45° C. After spraying, the granules were further dried at 30-50° C. for about 5-10 min until obtaining a final moisture below 5%. Acid resistance: 72%. 2 h neutral release: 66%.
Sample 4c: 300 g of enzyme granules of Control 1 were coated with an ethanolic solution of sorbic acid and ethyl cellulose, with a ratio 8:2, until reaching a weight gain equal to 25%. Oleic acid was used as plasticizer for ethyl cellulose with a ratio 1:8.2. The process was carried out using a fluid bed with an air temperature between 25 and 45° C. After spraying, the granules were further dried at 30-50° C. for about 5-10 min until obtaining a final moisture below 5%. Acid resistance: 97%. 2 h neutral release: 35%.
Sample 4d: 300 g of enzyme granules of Control 1 were coated with an ethanolic solution of sorbic acid and ethyl cellulose, with a ratio 7:3, until reaching a weight gain equal to 33%. Oleic acid was used as plasticizer for ethyl cellulose with a ratio 1:8.2. The process was carried out using a fluid bed with an air temperature between 25 and 45° C. After spraying, the granules were further dried at 30-50° C. for about 5-10 min until obtaining a final moisture below 5%. Acid resistance: 74%. 2 h neutral release: 33%.

### Example 5

Example 5 assessed the pelleting stability of the coated and uncoated products. Coated granules of Sample 1c and Control 1 were diluted with a carrier in order to achieve a target activity of 12 kU/g (measured with Azocasein, S-AZCAS, Megazyme Ltd). The enzyme was then included in a feed matrix, having a composition according to Table 1, at 3000 g/ton.

TABLE 1
Diet composition used for pelleting trial

| Ingredients | % |
|---|---|
| Corn | 72.36 |
| Soya bean | 25.4 |
| Calcium Carbonate | 1 |
| Sodium Chloride | 0.5 |
| Methionine DI | 0.31 |
| NaCl BUFFERVIT17 | 0.25 |
| L-Lysine | 0.18 |

Feed pellets were produced with a pelleting mill (CLM2005, La Meccanica s.r.l. di Reffo, Cittadella, Padova, Italy), equipped with a conditioner system allowing the injection of steam on the feed and a die-roll with 3.5 mm diameter holes. Conditioning was done at a controlled temperature of 85° C. for a duration of 30 seconds. After pelleting, enzyme activity was measured with Azocasein tablets (Protazyme AK, Megazyme Ltd).

Recovered enzyme activity for product from Sample 1c: 90%.

Recovered enzyme activity for product from Control 1: 38%.

It should be appreciated that without pore former, applying a low amount of coating (10%) the layer resulted with poor consistency, releasing most of the active, while increasing the coating amount (≥20%) the layer was more consistent, but poor releases were observed.

It should be evident to a person of ordinary skill in the art that for each type of active compound and depending on the morphology of the material to be coated, the matrix:pore former ratio and the weight gain of the coating should be optimized for the specific product.

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. A coating for a solid dosage form, comprising:
a first coating composition which comprises a hydrophobic matrix comprised of one or more fatty acids and a pore-forming compound comprising one or more weak organic acids, wherein the weak organic acid is protonated at a pH of about 3 or lower.

2. The coating of claim 1, wherein the weak organic acid is deprotonated at a pH of about 6 or higher.

3. The coating of claim 1, wherein the hydrophobic matrix comprises about 40 to 99 percent by weight of the first coating composition and the one or more weak organic acids comprise between about 1-60 percent by weight of the first coating composition.

4. The coating of claim 1, wherein the one or more fatty acids have a chain length of between C10 and C25.

5. The coating of claim 1, wherein the one or more fatty acids have a chain length of between C14 and C22.

6. The coating of claim 1, wherein the one or more fatty acids are selected from the group consisting of saturated, unsaturated, polyunsaturated, and mixtures thereof.

7. The coating of claim 1, wherein the one or more fatty acids have double bonds, said double bonds being cis or trans.

8. The coating of claim 1, wherein the one or more fatty acids is stearic acid.

9. The coating of claim 1, wherein the one or more weak organic acids is selected from the group consisting of alkyl carboxylic acids, alkenyl carboxylic acids, aryl carboxylic acids and mixtures thereof.

10. The coating of claim 1, wherein the one or more weak organic acids is selected from the group consisting of mono, di, tri, or poly carboxylic acids and mixtures thereof.

11. The coating of claim 1, wherein the one or more weak organic acids is selected from the group consisting of sorbic acid, benzoic acid, gallic acid, citric acid, lactic acid, and mixtures thereof.

12. The coating of claim 1, wherein the hydrophobic matrix and the pore-forming compound are soluble in an organic solvent.

13. The coating of claim 1, further comprising a second coating composition that forms a heat-resistant layer.

14. The coating of claim 13, wherein the second coating composition comprises HPMC.

15. The coating of claim 1, wherein the solid dosage form comprises a base component that is selected from the group consisting of a pharmaceutical, a nutraceutical, an enzyme, or a food or feed product.

16. The coating of claim 1, wherein the first coating composition does not include a plasticizer.

17. The coating of claim 1, wherein the first coating composition does not include an emulsifier.

18. The coating of claim 1, wherein the first coating composition is an enteric coating.

19. A method of coating a solid dosage form, the method comprising:
applying a mixture comprising one or more fatty acids and one or more weak organic acids to a solid dosage form to make a coated solid dosage form.

20. The method of claim 19, further comprising heating the mixture to solubilize the one or more fatty acids and one or more weak organic acids.

21. The method of claim 19, further comprising drying the coated solid dosage form.

22. The method of claim 19, further comprising applying a heat-resistant layer to the solid dosage form.

23. The method of claim 19, wherein the one or more fatty acids comprise about 40-99 percent by weight of the mixture and the one or more weak organic acids comprise between about 1-60 percent by weight of the mixture.

24. A method of stabilizing a solid dosage form for acidic conditions, comprising:
coating the solid dosage form with a mixture comprising one or more fatty acids and one or more weak organic acids.

25. The method of claim 24, wherein the one or more fatty acids comprise about 40-99 percent by weight of the mixture and the one or more weak organic acids comprise between about 1-60 percent by weight of the mixture.
